(19) United States
(12) Patent Application Publication
(10) Pub. No.: US 2014/0114283 A1
(43) Pub. Date: Apr. 24, 2014

(54) NURSE CONTROLLED ACCESS OF MEDICATION PUMP

(71) Applicant: Cory Congleton, Lambertville, MI (US)

(72) Inventor: Cory Congleton, Lambertville, MI (US)

(21) Appl. No.: 13/658,772

(22) Filed: Oct. 23, 2012

(51) Int. Cl. A61M 1/00

(52) U.S. Cl. 604/507

(57) ABSTRACT

A method of distributing medication is disclosed herein. The method includes the step of opening a fluid pathway for delivering fluid intravenously to a patient. The method also includes the step of placing a medication pump in fluid communication with the pathway. The method also includes the step of locking a user interface of controls for the medication pump to place the medication pump under the exclusive control of medical professionals.

### BACKGROUND OF THE INVENTION

1. Field of the Invention

The invention relates to the control of apparatus for introducing material to the body for therapeutic purposes, such as medicating.

2. Description of Related Prior Art

U.S. Pat. No. 7,967,773 discloses a two piece medication cassette closure apparatus and method. The device may include a first assembly comprising a rear housing and a pressure plate affixed to one another, as well as a second assembly comprising a cover that includes a plurality of lock feature protrusions and at least one slotted aperture. The first assembly and second assembly are adapted for permanent coupling by first and second lock arrangements. Generally, the first lock arrangement includes lock feature protrusions of the second assembly engaging within the lock receiving structures of the first assembly. Further, the second lock arrangement includes at least one tabbed snap member of the first assembly engaging within at least one corresponding slotted aperture of the second assembly.

### SUMMARY OF THE INVENTION

In summary, the invention is a method of distributing medication. The method includes the step of opening a fluid pathway for delivering fluid intravenously to a patient. The method also includes the step of placing a medication pump in fluid communication with the pathway. The method also includes the step of locking a user interface of controls for the medication pump to place the medication pump under the exclusive control of medical professionals.

### BRIEF DESCRIPTION OF THE DRAWING

Advantages of the present invention will be readily appreciated as the same becomes better understood by reference to the following detailed description when considered in connection with the accompanying drawing wherein:

FIG. 1 is a schematic illustration of an embodiment of the invention.

### DETAILED DESCRIPTION OF EXEMPLARY EMBODIMENT

The inventor has perceived that in the field of medication distribution doctors may prescribe medication at different dosage volumes and different rates for different patients. This pain medication can be delivered intravenously. The delivery of medication to the patient is not controlled exclusively by the doctor's proscription. The nurse will exercise professional judgment by evaluating the patient's condition and might forego the delivery of a dosage of medication, such if the patient's rate of respiration is below some predetermined rate.

Medication can be withdrawn from a computerized medication dispenser by the nurse. If the volume of the required dosage is different than the volume of medication available in the dispenser, the inventor has perceived that substantial inefficiencies arise. If the volume of medication in the dispenser is less than the quantity required, the medication in the dispenser must be expelled. This quantity of medication is thus wasted. Further, the dispenser must then be filled before the patient's medication can be obtained and treating the patient is delayed. Also, if the available stock in the dispenser is larger than the ordered dose of medication, the excess medication must again be wasted. In addition to the issue of waster, the efficiency of the process is also compromised by the fact that a second nurse must be present to witness the disposal of the excess medication. The dedication of two nurses to this activity detracts from the care of other patients. If the second nurse is not available, the first nurse is not able to access the medication and the patient's suffering is prolonged.

In one example, a patient who has severe pain may be prescribed intravenous pain medication at higher frequencies of administration, such as every 5 minutes, 10 minutes, 15 minutes, 1 hour, or 2 hours per intravenously-administered dose. This situation can arise after major surgery, in cancer patients, palliative care, and in hospital hospice patients. This level of acuity in this type of care setting is demanding and sometimes difficult to safely and effectively handle, depending on the number of other patients that a nurse is monitoring. The condition of these patients may not be classified as unstable so these patients are not sent to the Intensive Care Unit (ICU) where the nurses usually have a lower patient ratio. The patient is usually sent to medical surgical type unit with RNs having high patient ratios. Embodiments of the invention could have profound impact in these operating environments.

In another example of practicing the embodiment, a doctor might order morphine sulfate, IVP 4 mg every 5 min, as needed. The nurse might be directed to hold medication if the patient's rate of respiration is below eight breaths per min. At this frequency and dosage, the nurse would have to find a second nurse to witness and document activities in the computerized medical dispenser up to twelve times per hour. An embodiment of the present invention would allow the nurse to only have to find a second nurse to activities in the computerized medical dispenser when the new cartridge for the medication pump is initiated, when the cartridge is empty, or when the order is discontinued. Thus, the twelve, two-nurse events per hour could be reduced to less than one (on average). This saves nursing time, provides more timely availability to the patient's needs, decreases medication waste, and decreases patient's risk of infection by decreasing number of times the nurse would have to access intravenous port for the multiple medication dosage administrations.

Pumps that can deliver metered dosages of medication to a patient can deliver medication to the intravenous flow path. Typically, the operation of such pumps is controlled in part by the patient and in part by the logic programming of the pump's controller. For example, a pump can be programmed to allow a patient to release a quantity of medication after a predetermined period of time has passed since the last dosage. The patient may choose to forego the dosage and therefore not engage the pump.

Current regulations dictate that only the patient is permitted to exercise control over the operation of the pump. These regulations are intended to preclude family members, for example, from activating the pain pump when the patient is unable to do so. This situation might arise if the patient's rate of respiration is relatively low and/or the patient is unconscious. If the patient is in such a condition, additional pain medication could be harmful.

In the present invention, a medication pump is maintained under the control of a nurse. The pump can be controlled by the nurse by physical means, such as a lockbox. The nurse will use the pump to deliver medication instead of the computerized medication dispenser. The medication capacity of pumps is greater than the medication capacity of computerized medication dispenser. As a result, the frequency of medication replacement is reduced. This, in turn, will result is less medication waste and fewer instances of two nurses being dedicated to the care of a single patient.

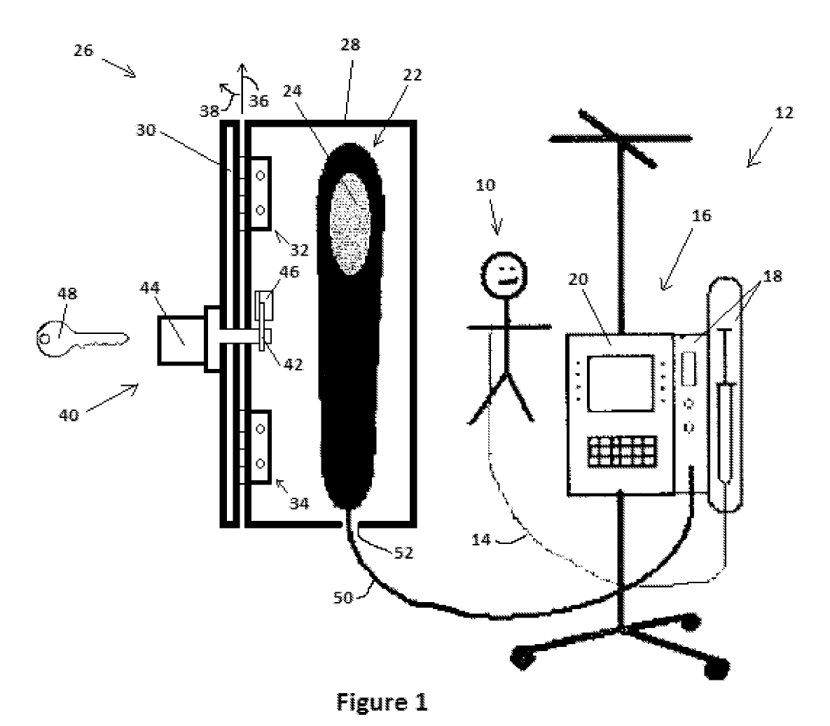

FIG. 1 is a schematic illustration of an embodiment of the invention. A patient 10 in need of medication has been connected to a medication delivery system 12 by opening a fluid pathway for delivering fluid intravenously to the patient 10. The medication delivery system 12 includes an intravenous line 14 disposed to access the patient. The medication delivery system 12 also includes a medication pump 16. The exemplary medication pump can include pump module 18 or cassette containing medication and a primary module 20 operable to delivery mechanical or electrical energy to drive pumping of the medication.

The intravenous line 14 is the medium for intravenous medication administration from the medication pump 16. The medication delivery system 12 thus places the medication pump 16 in fluid communication with the pathway for delivering fluid intravenously to the patient 10.

The medication pump 16 also includes a control module 22. The exemplary control module 22 includes a button 24 that can be pressed to activate pumping and the delivery of medication through the intravenous line 14. In the exemplary embodiment, the delivery of medication by the medication pump 18 through the intravenous line 14 will not occur unless the button 24 is pressed. In the exemplary embodiment of the invention, the control module 22 defines user interface of controls for the medication pump 16.

The exemplary control module 22 is locked to place the medication pump under the exclusive control of medical professionals, such as nurses. In the exemplary embodiment of the invention, the control module 22 is disposed in a lockbox 26. The lockbox 26 includes a box portion 28 and a lid 30 connected together by hinges 32 and 34. The lid 30 can pivot between open and closed positions about a pivot axis 36 of the hinges 32 and 34. The pivoting movement is represented by arrow 38.

The lockbox 26 can also include a lock mechanism 40. The lock mechanism 40 can include a rotatable arm 42 and key barrel 44 for locking the arm 42 by preventing rotation of the arm 42, both mounted in the lid 30. The lock mechanism 40 can also include a catch 46 mounted in the box portion 28. A key 48 for unlocking the arm 42 and allowing movement of the lid 30 can be kept by a nurse. It is noted that a wire 50 for placing the control module 22 in electric communication with the primary module 20 can extend through an aperture 52 in the lock box 26.

The exemplary embodiment can be crafted from a transparent lexan polycarbonate. Approximate dimensions can be 2 inches in height, 2 inches in width, and 5 inches in length. The box portion can be formed from a plurality of pieces cut with a circular saw and bonded together with a high strength epoxy. The catch can be bonded to the inside wall of the box portion by epoxy glue. The holes that fasten the hinges to the box can be predrilled. It is noted that these construction and dimension details are offered as examples and are not limiting on the broader invention.

In practicing the exemplary embodiment, a hospital or other care-giving facility can direct employee(s) to open a fluid pathway for delivering fluid intravenously to a patient. This can be accomplished by establishing an intravenous port in the patient's body. The direction can be provided in the form of policies and guidelines applicable to the care to be given to patients that nurses and other care providers are obliged to follow. Alternatively, the direction can be specialized for a particular patient and be given by one employee of the hospital or other care-giving facility (such as a doctor or supervisory nurse) to another employee of the hospital or other care-giving facility (such as a nurse). The hospital or other care-giving facility can then direct employee(s) to place a medication pump in fluid communication with the pathway. This can be accomplished by connecting the medication pump to the a fluid line in fluid communication with the intravenous port so that fluid pumped by the medication pump travels to the intravenous port through the fluid line (or intravenous line) and into the patient's body. Again, the direction can be provided in the form of policies and guidelines applicable to the care to be given to patients that nurses and other care providers are obliged to follow. The hospital or other care-giving facility can also direct employee(s) to lock a user interface of controls for the medication pump to place the medication pump under the exclusive control of medical professionals, such as employee(s) of the hospital or other care-giving facility. In exercising patient care, a nurse can assess a patient and determine that providing a dose medication with the medication pump is appropriate. The nurse can unlock the user interface of controls for the medication pump, such as by removing the controls from a locked container or by providing fingerprints for identity confirmation. After the controls of the pump are accessible, the nurse can control the medication pump to deliver the dosage and then return the controls to a locked condition, either a physical locking or logic/electric locking arrangement.

The invention is universal and applicable to all medication pumps currently viewed as patient-controlled. Practicing the invention does not require physical modification of an existing medication pumps or modification function. The invention is adaptive and compatible with all known the patient-controlled medication pump remotes.

As set forth above, the pump can be controlled by being placed in a lockbox, wherein a nurse possesses the key. It is also noted that control over the operation of the medication pump can be exercised in other ways. Some medication pumps accommodate finger print scanning and an embodiment of the invention can be practiced wherein a nurse's fingerprint is designated as the fingerprint allowing access to the controls of medication pump.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Further, the “invention” as that term is used in this document is what is claimed in the claims of this document. The right to claim elements and/or sub-combinations that are disclosed herein as other inventions in other patent documents is hereby unconditionally reserved.

What is claimed is:

1. A method of distributing medication comprising the steps of:
opening a fluid pathway for delivering fluid intravenously to a patient;
placing a medication pump holding medication in fluid communication with the pathway;
administering the medication to the patient by manipulating a user interface of controls of the medication pump to activate the medication pump, such that the medication is placed in the fluid pathway by pumping action of the medication pump; and
locking the user interface of controls of the medication pump to place the medication pump under the exclusive control of medical professionals.

2. The method of claim 1 wherein said locking step is further defined as:
placing the medication pump in lock box such that a user interface of controls for the medication pump are inaccessible when the lock box is in a locked configuration.
